(19) United States
(12) Patent Application Publication
(10) Pub. No.: US 2004/0157800 A1
(43) Pub. Date: Aug. 12, 2004

(54) Ascorbyl derivatives of carnitines and cosmetic compositions containing same

(76) Inventors: Antonietta Buononato, Rome (IT); Emanuela Veggetti, Brescia (IT)

Correspondence Address: Nixon & Vanderhye, 8th Floor, 1100 North Glebe Road, Arlington, VA, 22201-4714, US

(21) Appl. No.: 10/481,796

(22) PCT Filed: Jun. 17, 2002
(86) PCT No.: PCT/IT02/00399

(30) Foreign Application Priority Data
Jun. 25, 2001 (IT) RMK01A000364

(51) Int. Cl. A61K 31/665

(52) U.S. Cl. 514/100; 549/218

(57) ABSTRACT

Novel L-carnitine and lower alkanoyl L-carnitine ascorbyl derivatives and topically applicable cosmetic compositions comprising same as active ingredients. The cosmetic compositions are particularly suitable for preventing and treating the anaesthetic consequences of localized adiposity and the cascade of dysmetabolic events linked thereto.

The present invention relates to novel derivatives of L-carnitine and lower alkanoyl L-carnitines and the cosmetic compositions comprising such derivatives as active ingredients.

More particularly, the present invention relates to stable non-hygroscopic ascorbyl derivatives of L-carnitine and lower alkanoyl L-carnitines which, following intense absorption through the skin, exert the favourable physiologic activity of both vitamin C and that peculiar of carnitines, in the dermis and underlying tissues.

By “lower” alkanoyl L-carnitines are meant those compounds wherein the straight or branched-chain alkanoyl group contains 2-5 carbon atoms. Preferred examples of alkanoyl groups are acetyl, propionyl and isovaleryl.

The topically applicable cosmetic compositions according to the invention are particularly effective in the prevention and treatment of localized adiposity, panniculitides and, particularly, aedemato-liposclerotic panniculitis, i.e. the excessive formation of subcutaneous panniculus adiposus, a condition which is commonly and improperly called “cellulite”.

The use of L-carnitine and derivatives thereof (particularly, L-carnitine salts) in the cosmetic field has long since been known.

U.S. Pat. No. 4,839,159 (Topicarn Inc.) discloses L-carnitine-containing topical compositions useful for preventing, improving or healing a number of skin conditions related to loss of elasticity and epidermal exfoliation. These skin conditions comprise wrinkling, dryness, scarring such as that caused by chickenpox and burns, particularly those due to excessive exposure to sunlight.

U.S. Pat. Nos. 5,591,450; 5,614,556 and 5,637,305 (Avantgarde S.p.A.) relate to L-carnitine salts with, respectively, glycolic, trichloroacetic and azelaic acid, useful as active ingredients of pharmaceutical cosmetic compositions suitable for the treatment of dermatosis such as ichthyosis, psoriasis, dandruff, palmar and plantar hyperkeratosis.

All the previously mentioned cosmetic applications of L-carnitine and derivatives thereof relate to the treatment of various disorders of epidermis, not the underlying tissues. In particular, the use of L-carnitine or salts thereof has never been proposed with a view to treating panniculitides and, especially, aedematoliposclerotic panniculitis.

Also the basic physiologic functions of vitamin C (ascorbic acid) are well known (see e.g. Goodman and Gilman's “The pharmacological basis of therapeutics”, Eight Edition, 1990, pages 1547-1552).

The functions of vitamin C related to the effects it exerts on the skin are mainly bound to the synthesis of collagen and elastin and, due to its known antioxidant properties, to the prevention of melanogenesis which is responsible of the formation of melanin spots on epidermis.

However, vitamin C is an unstable compound, barely absorbable through the skin.

In order to overcome these drawbacks, a vitamin C derivative, magnesium ascorbyl phosphate, has been developed which, in contrast with free ascorbic acid, is characterized by excellent stability both in neutral and slightly alkaline environments.

This compound is further characterized by its ability to penetrate the skin as far as the epidermis melanocytes and release to their cytoplasm the ascorbic acid which is apt to take up the oxygen at the melanosoma level thus preventing tyroxine oxidation.

In connection with this useful ascorbic acid derivative, too, no activity against the excessive formation of subcutaneous panniculus adiposus has ever been reported.

To-date, the only effective means for counteracting the subcutaneous panniculus adiposus has consisted in liposuction, a surgical procedure based on the removal of fatty plethora by selective breakdown and suction thereof.

No one of the various cosmetic preparations available on the market, which advertize an alleged “anti-cellulite” activity has ever shown an efficacy supported by scientifically corroborated pharmacological and/or clinical tests.

It has now been found that the L-carnitine and alkanoyl L-carnitine magnesium ascorbyl phosphates of formula (I)

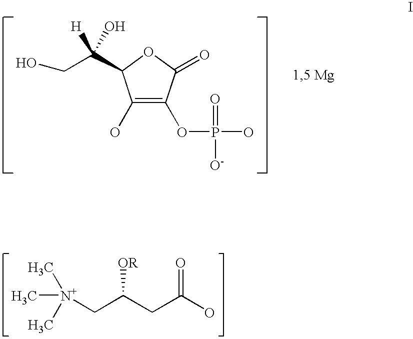

wherein R is hydrogen or a straight or branched-chain lower alkanoyl having 2-5 carbon atoms, preferably selected from acetyl, propionyl and isovaleryl, are stable and non-hygroscopic compounds suitable to be absorbed intensely through the epidermis and deeply penetrate the underlying structures as far as the adipocyte-rich fat tissue, where they perform a potent scavenging action on acyl radicals.

The compounds of formula (I) are, therefore, effective active ingredients of topically applicable cosmetic compositions such as creams, ointments, gels, suspensions, lotions, emulsions and the like, suitable for preventing or treating the excessive formation of subcutaneous panniculus adiposus.

The following non-limiting examples illustrate the preparation and physico-chemical characteristics of some compounds according to the invention.

### EXAMPLE 1

### L-Carnitine Magnesium Ascorbyl Phosphate (BS/220)

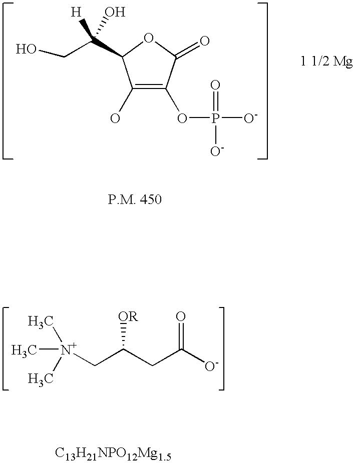

28.95 g (0.1 moles) of magnesium ascorbyl phosphate and 16.1 g (0.1 moles) of L-carnitine inner salt were dissolved in 85 mL of water at 50° C. and the resulting solution slowly poured into an alcoholic solution consisting of 350 mL of isopropanol under vigorous mechanical stirring. A solid precipitated which was filtered off and dried. 43 g of L-carnitine magnesium ascorbyl phosphate were obtained (yield 96%) as a white, crystalline, non-hygroscopic solid.

Melting point=147-148° C.

[α]^{D}₂₅=+15.9(c=1% H₂O)

pH=7.5 (c=1% H₂O)

NMR: D₂O=4.5-4.4 (1H,m,

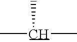

); 4.45-4.3 (1H,d,

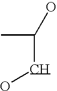

); 4.1-3.9 (1H, q, CH—CH₂—OH); 3.7-3.6 (2H, d, CH₂—OH); 3.4-3.35 (2H, d, CH₂—N); 3.2 (9H, s, (CH₃)₃—N); 2.4-2.3 (2H, q, CH₂—COOH)

| Elementary | | | | |
|---|---|---|---|---|
| analysis | C % | H % | N % | Mg % |
| Calculated: | 34.5 | 4.9 | 3.1 | 8 |
| Found: | 33.98 | 4.71 | 3.07 | 7.98 |

HPLC analysis:

Column: SPHE-SAX 5 μm 250×4.6 mm; Temperature: 30° C. Eluant: water+0.1M CH₃CN/KH₂PO₄ (65/35); pH=5.5; Flow-rate: 1 mL/minute; Rt=L-Carnitine: 10 minutes; Rt=Magnesium ascorbyl phosphate: 18 minutes; Ratio: L-Carnitine=35%; Ascorbyl phosphate 57%; Magnesium 8%.

### EXAMPLE 2

### Acetyl L-Carnitine Magnesium Ascorbyl Phosphate (BS/230)

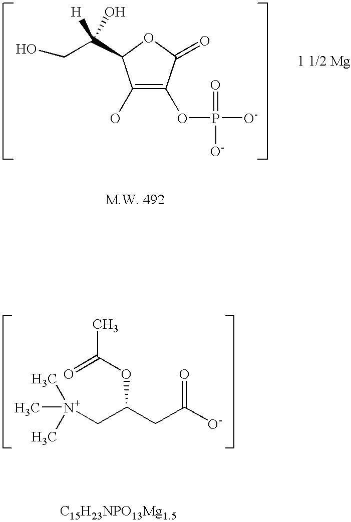

28.95 g (0.1 moles) of magnesium ascorbyl phosphate were dissolved in 50 mL of water at 50° C. (solution A) and 20.3 g (0.1 moles) of acetyl L-carnitine inner salt were dissolved under heating in an alcoholic solution consisting of 300 mL of ethanol (solution B).

The solution A was poured into solution B under vigorous mechanical stirring. A solid precipitated which was filtered off and dried. 45 g of acetyl L-carnitine magnesium ascorbyl phosphate were obtained (yield 93%) as a white, crystalline, non-hygroscopic solid.

Melting point: 261-263° C.

[α]^{D}₂₅=+18.1 (c=1% H₂O)

p.H. =7.1 (1% aqueous solution)

| Elementary | | | | |
|---|---|---|---|---|
| analysis | C % | H % | N % | Mg % |
| Calculated: | 36.5 | 4.67 | 2.84 | 7.3 |
| Found: | 36.1 | 4.59 | 2.81 | 7.28 |

NMR: D₂O=5.5-5.4 (1H, m,

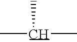

); 4.45-4.3 (1H, d,

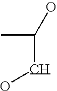

); 4.1-3.9 (1H, q, CH—CH₂—OH); 3.8-3.5 (2H, m, CH₂N); 3.7-3.6 (2H, d, CH₂—OH); 3.25-3.15 (9H, s, (CH₃)₃N); 2.65-2.55 (2H, m, CH₂COOH); 2 (3H, s, COCH₃)

HPLC analysis:

Column: SPHE-SAX, 5 μm 250×4.6 mm; Temperature: 30° C.

Eluant: water+0.1 M CH₃CN/KH₂PO₄ (65/35); pH=5.5

Flow-rate: 1 mL/minute; Rt=acetyl L-carnitine: 7.4 minutes

Rt=magnesium ascorbyl phosphate: 24 minutes

Ratio: acetyl L-carnitine=41%

ascorbyl phosphate=52%

magnesium=7.3%

In the following Table 1, the weight increase (%) and the flowability of the compounds of the invention are compared with those of L-carnitine and acetyl L-carnitine inner salts following exposure of the compounds to 60% relative humidity at 25° C. for 42 hours. Reference method:

Pharmaeuropa November 1996.

TABLE 1

| | Weight | | |
|---|---|---|---|
| Compound | increase (%) | Appearance | Flowability * |
| L-carnitine | 19 | deliquescent | |
| Inner salt | | | |
| acetyl L- | 20 | deliquescent | |
| carnitine | | | |
| Inner salt | | | |
| Compound of | 0.12 | no variation | 5 seconds |
| Example 1 (BS220) | | | |
| Compound of | 0.14 | no variation | 6 seconds |
| Example 2 (BS230) | | | |

* The flowability is given by the time (in seconds) needed by a 10-gram sample of the compounds to pass through a funnel having diameter of 4 mm.

The compositions according to the invention may comprise further active ingredients such as, e.g., substances endowed with lipolytic activities. Among these substances, the extract of phytoderivatives from the peel of Citrus aurantium amara which, in combination with the ascorbyl derivatives of the present invention, develops a potent synergistic effect, is preferred.

Citrus aurantium amara (bitter orange), an evergreen tree native of Southern China and North-East India, is nowadays cultivated in China, Southern Europe and the United States where, because of its sturdiness and resistance to pathogenic bacteria, is used as a stock for sweet oranges.

The bitter orange oil is obtained from fresh epicarps by cold squeezing.

In addition to the volatile oil (1-25%), more than 90% of which is comprised of monoteipenes (mainly limonene), the bitter orange epicarp contains considerable amounts of neohesperidin (up to about 14% in unripe epicarps, currently 2.4-2.8% in ripe epicarps), naringin (0.9-4%), roifolin, lonicerin, hesperidin and further flavonoids (tangeretiu, nobiletin, sinensetin, aurantiin, rutin), vitamins (A, Bi and C); coumarins (6,7-dimethoxycoumarin and umbelliferone); carotenoid pigments (citraurin, violaxanthin and cryptoxanthin); pectin and citrantin.

In folk medicine, particularly in Chinese folk medicine, the dried peel of bitter orange is used as tollic, as carminative in the treatment of dyspepsia and in the treatment of descensus uteri and diarrhea.

Recently, its potent lipolytic activity has been shown. This is brought about by the presence of substances having sympathomimetic activity (particularly synephrine) which, binding to the adrenergic P3 receptors of fatty tissue, trigger the localized release of adrenalin to which the lipolytic effect can be attributed.

### In Vitro Tests

In this study, 3T3 L1 embryonal fibroblasts were used [Green H., Kehinde O. (1974) Cell 1: 113-116].

These fibroblasts are a cell-line whose main characteristic is the ability to accumulate lipids, particularly triglycerides, in their cytoplasm. Provided they are kept in confluent culture in the presence of high concentrations of serum, the fibroblasts are liable to differentiate to adipocytes, in the cytoplasm of which accumulation of triglycerides in the form of “droplets” can be observed.

This cell-line was cultivated in sterile flasks (T75) incubated at 37° C. in a 5% CO₂ humid air and with 15 mL EMEM (Eagle's Minimum Essential Medium in Earle's BSS) culture medium added with 10% fetal calf serum (FCS), 1 mM sodium pyruvate and in the presence of 100 U/mL of penicillin and 100 μg/mL of streptomycin as antibiotics.

The 1:2 split was carried out every five days upon reaching monolayer formation by washing with PBS 1X (Ca⁺⁺ and Mg⁺⁺-free phosphate buffer) and cell detachment with a 0.25% trypsin solution in EDTA, at 37° C. for 5 minutes.

An oil-in-water (o/w) emulsion was prepared containing 2.5% of phytoderivative extract from Citrus aurantium amara [E F D C A] and 1.5% of the compound of Example 1.

In order to assess the maximum non-toxic dose of the o/w emulsion, cells undergoing exponential growth were detached with 0.25% trypsin-EDTA and suspended again in the culture medium so as to obtain a single cell suspension; they were then plated on sterile 96-well plates (ELISA plates) at the concentration of 10 cells/100 μL of culture medium for each well.

Following 24 hour incubation, at 37° C., in 5% CO₂ humid air, the culture medium was replaced with 100 μL of emulsion to be tested at increasing concentrations in active ingredient (the compound of Example 1). See Table 2.

All dilutions were performed in the culture medium.

Following 24-hour exposure to the topic agent, the emulsion toxicity was assessed by means of the test which utilizes MTT (3-[4.5-dimethylthiazol-2 yl]-2.5-diphenyltetrazolium bromide) (Roche) [Mosmann T.(1988) J. Immunol. Methods 65:55-63].

This method is based on the ability of the actively proliferating cells to break the MTT's tetrazole ring by NADH— and NADPH— dependent reduction, thus leading to formazane formation which precipitates in the culture medium in the form of blue crystals.

The addition of a solubilizing agent (10% SDS,0.01M HCl) dissolves the crystals and allows the assessment of the colour intensity to be carried out by 540 nm absorbance.

After 24-hour contact, 10 μL of MTT at the final concentration of 0.5 mg/mL were added to each well which contained already the culture medium and the compound to be tested.

Following 4-hour incubation at 37° C. in 5% CO₂ humid air, 100 μL of solubilizing solution were added to each well.

After one night at 37° C., the absorbance at the wavelenght of 540 nm was measured by means of a microplate reader (Biorad). The non-toxic dose was then assessed on 8 replica for each dilution (see Table 2), by reckoning the % Relative Growth Rate (% RGR) according to the formula:

$$\% RGR = \frac{Number of treated cells}{Number of untreated cells} \times 100$$

TABLE 2
MTT Test

| | | | |
|---|---|---|---|
| | Mean | | |
| Absorbance | | | |
| Concentration | (OD) | Std. Dev.(1) | % RGR(2) |

| | | | | |
|---|---|---|---|---|
| | | 0.535 | 0.022 | 100.000 |
| 500 | μg/mL | 0.189 | 0.014 | 35.33 |
| 50 | μg/mL | 0.577 | 0.028 | 107.85 |
| 5 | μg/ml | 0.523 | 0.019 | 97.76 |
| 500 | ng/mL | 0.513 | 0.022 | 95.89 |
| 50 | ng/mL | 0.523 | 0.029 | 97.76 |
| 5 | ng/mL | 0.522 | 0.021 | 97.57 |
| 500 | pg/mL | 0.530 | 0.055 | 99.06 |

(1)standard deviation
(2)relative growth rate (%) = number of treated cells/number of untreated cells (%)

The emulsion's lipolytic activity was tested on the 3T3L1 cell-line by the following method: 1.5×10⁵ cells/500 μL of culture medium were seeded in each well of Labtek II Chamber Slides.

After 24-hour incubation at 37° C. in 5% CO₂ humid air, the 3T3L1 cells were contacted with both the previously assessed non toxic doses and 10 U lipase (positive control) for 24 hours.

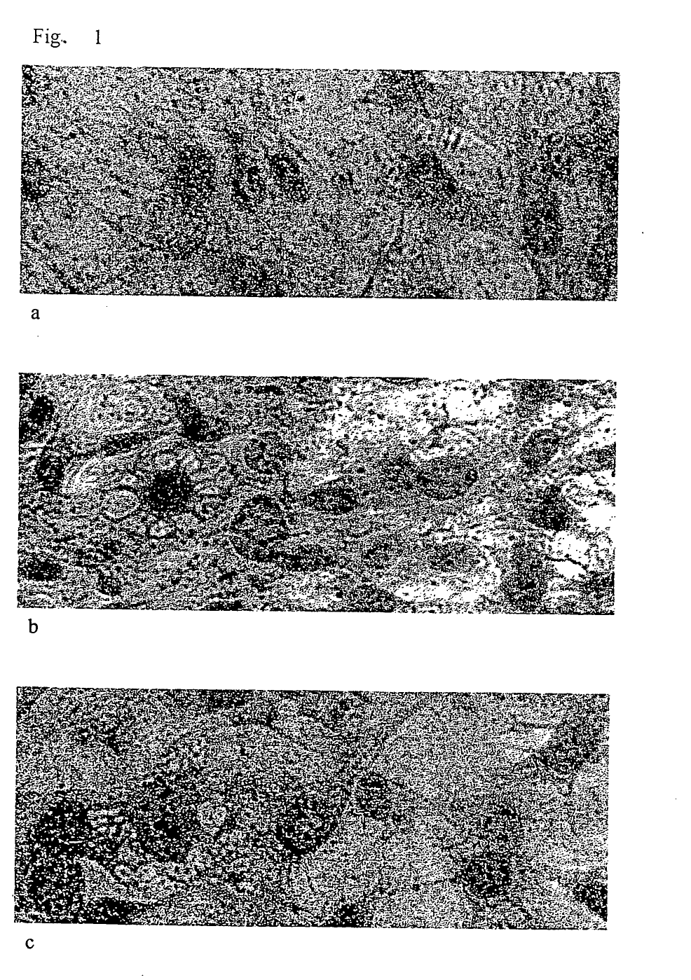

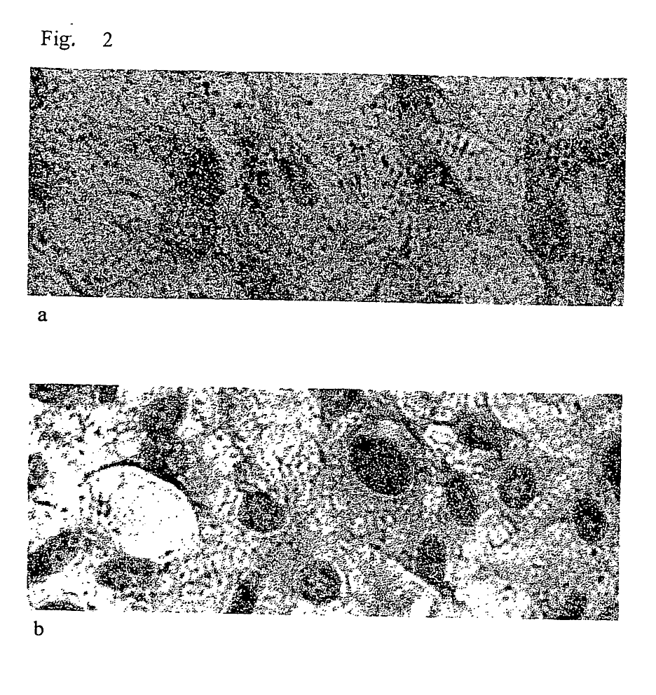

The cells were then washed twice with PBS1X and fixed by addition of 4% paraformaldehyde in PBSLX in the dark, at room temperature. The cells were then stained with Oil Red O (specific staining for triglycerides) for 1 hour and then stained again with Harris haematoxiline (specific staining for nuclei) for 10 minutes [Preece A. (1972) Manual for Histology Technicians: 260 (Boston: Little, Brown of Co.)]. Following stain removal, the cells were studied with a phase-contrast microscope (Nikon Eclipse TE-200) and photographed with Kodak film (FIGS. 1 and 2).

FIG. 1 shows (530× magnification)

in the frame A, untreated adipocytes

in the frame B, adipocytes treated with the emulsion at the dose of 50 μg/mL; and

in the frame C, adipocytes treated with the emulsion at the dose of 500 μg/mL.

FIG. 2 shows (530× magnification)

in the frame A, untreated adipocytes; and

in the frame B, adipocytes treated with lipase (10 U).

By comparing the various frames, it can be noticed that the adipocytes inclusions consisting of fatty droplets are remarkably decreased. This shows the splitting of fats (triglycerides) and the removal of hydrolysis products (fatty acids).

Therefore, the adipocytes turn out to be depleted of their contents, the determining factor of pathogenesis and initial event of panniculitis. In fact, it should be recalled that an excess of fats (in this specific case, at the level of subcutaneous adipose tissue) brings about an increase in the number of adipocytes (hyperplasia) and size thereof (hypertrophy).

1. L-carnitine or lower alkanoyl L-carnitines ascorbyl phosphate of formula 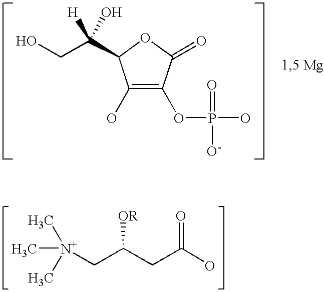
wherein R is hydrogen or a straight or branched-chain alkanoyl having 2-5 carbon atoms.

2. The ascorbyl phosphate of claim 1 wherein the alkanoyl is selected from the group comprising acetyl, propionyl and valeryl.

3. L-carnitine magnesium ascorbyl phosphate.

4. Acetyl L-carnitine magnesium ascorbyl phosphate.

5. A topically applicable composition comprising as active ingredient at least one L-carnitine or lower alkanoyl L-carnitine ascorbyl phosphate of formula 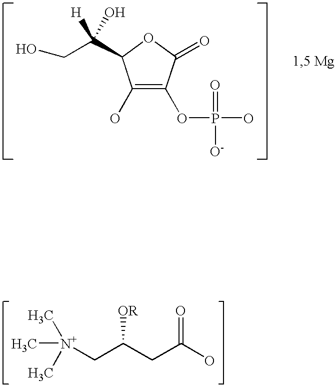
(wherein R is hydrogen or straight or branched-chain alkanoyl having 2-5 carbon atoms).

6. The composition of claim 5 in the form of cream, ointment, gel, suspension, lotion or emulsion.

7. The composition of claims 5 or 6 comprising an amount of said ascorbyl phosphate effective for preventing or treating the excessive formation of the subcutaneous panniculus adiposus.

8. The composition of claims 5 or 6 comprising an amount of said ascorbyl phosphate effective for preventing the onset of or treating a condition of panniculitide.

9. The composition of claim 8 for preventing the onset of or treating aedematoliposclerotic panniculitis.

10. The composition of claim 5-9, further comprising a lipolytic agent.

11. The composition of claim 10, wherein the lipolytic agent is an ex-tract of bitter orange (citrus aurantium amara) peel.

12. The composition of claim 11 which comprises from 0.2 to 2% w/w of ascorbyl derivative and from 0.5 to 2.5% w/w of citrus aurantium amara extract.
